(19) United States
(12) Patent Application Publication
(10) Pub. No.: US 2021/0350913 A1
(43) Pub. Date: Nov. 11, 2021

(54) COMPREHENSIVE DIGITAL HEALTHCARE PLATFORM

(71) Applicant: Comprehensive Healthcare Technology LLC, Northfield, IL (US)

(72) Inventor: Peter Leo Breen, Northfield, IL (US)

(73) Assignee: Comprehensive Healthcare Technology LLC, Northfield, IL (US)

(21) Appl. No.: 17/308,561

(22) Filed: May 5, 2021

Related U.S. Application Data
(60) Provisional application No. 63/020,744, filed on May 6, 2020.

(51) Int. Cl. G16H 40/20; G16H 10/60; G06Q 20/08

(52) U.S. Cl. CPC G16H 40/20; G06Q 20/0855; G16H 10/60

(57) ABSTRACT

Systems and methods for a comprehensive digital healthcare platform.

### PRIORITY

This application claims priority to U.S. provisional Patent Application No. 63/020,744, filed on May 6, 2020, which is incorporated herein by reference in its entirety.

### FIELD OF THE INVENTION

Embodiments of the present disclosure relate to systems and methods for transforming consumer health care experiences through the integration of multiple independent computer systems into a single platform on a mobile device, the embedding of a personal quick response (QR) code into the user's platform for real-time access and storage of medical data for the user, and the use of the QR code as a smart health card. The disclosed use of such a QR code can prove especially useful for hand-held devices in that the user can perform various interrelated functions (e.g., confirm medical eligibility, pricing, etc.) without the need to constantly log in and out of multiple computer systems to successfully perform various functions.

### BACKGROUND OF THE DISCLOSURE

Healthcare experiences are too complicated—consumers can benefit from the removal of the friction and frustration that currently exists in such experiences. Consumers want to be able to navigate and manage their healthcare experiences in a straightforward manner. Since Health Plans (or other similar type entities) are at the core of every single healthcare experience, they are the obvious builders of the right tools to address the gargantuan transaction processing waste and palpable consumer frustration.

### SUMMARY OF THE DISCLOSURE

Embodiments of the present disclosure relate to a digital healthcare system comprising a server communicably coupled to a user device associated with a user. The server being configured to obtain information associated with the user by: downloading benefits plan data and provider network contract details from a health plan server; downloading scheduling data from one or more provider devices; downloading medical results associated with the user and claims processing information from the health plan server; and downloading one or more third-party applications. The server being further configured to embed the information into a QR code transmitted to the user device; and establish one or more communication channels for the user device.

Embodiments of the present disclosure relate to a method, performed by a server communicably coupled to a user device associated with a user. The method comprises, among other things, obtaining information associated with the user by: downloading benefits plan data and provider network contract details from a health plan server; downloading scheduling data from one or more provider devices; downloading medical results associated with the user and claims processing information from the health plan server; and downloading one or more third-party applications. The method further comprises embedding the information into a QR code transmitted to the user device; and establishing one or more communication channels for the user device.

### BRIEF DESCRIPTION OF THE FIGURES

FIG. 1 is a block diagram of an example system for providing a comprehensive digital healthcare platform according to some embodiments of the present disclosure.

FIG. 2 shows an example transformative 3D health management QR code according to some embodiments of the present disclosure.

FIG. 3 is a flow diagram showing example processing that can launch a digital healthcare platform according to some embodiments of the present disclosure.

FIG. 4 is a flow diagram showing example processing that can be performed at a point-of-service according to some embodiments of the present disclosure.

FIGS. 5A and 5B show an example communication channel according to some embodiments of the present disclosure.

FIGS. 6A and 6B show another example communication channel according to some embodiments of the present disclosure.

FIGS. 7A and 7B show another example communication channel according to some embodiments of the present disclosure.

FIGS. 8A and 8B show another example communication channel according to some embodiments of the present disclosure.

FIGS. 9A and 9B show another example communication channel according to some embodiments of the present disclosure.

FIG. 10 is a block diagram illustrating some of the components of a user device and or provider device configured according to one embodiment of the present disclosure.

FIG. 11 illustrates some of the components of a device according to one embodiment of the present disclosure.

FIG. 12 is a block diagram illustrating some of the components of a server configured according to one embodiment of the present disclosure.

FIG. 13 is an example user interface that can be presented on a user device according to some embodiments of the present disclosure.

FIG. 14 is another example user interface that can be presented on a user device according to some embodiments of the present disclosure.

FIG. 15 is another example user interface that can be presented on a user device according to some embodiments of the present disclosure.

FIG. 16 is another example user interface that can be presented on a user device according to some embodiments of the present disclosure.

### DETAILED DESCRIPTION

The disclosed digital platform tool for Health Plans adds the value that consumers are looking for today—real functionality, positive & friendly experiences, and easy to use products. Many current Health Plans (or similar type entities) are not utilizing current technology to address their operational inefficiencies. The disclosed Comprehensive Healthcare Technology (CHT) platform incorporates technology to make healthcare experiences more efficient and to generate new data and information that otherwise would not be available to consumers and providers. The disclosed digital platform can offer user-side benefits that improve everyday healthcare experience by providing consumers and users with useful tools and meaningful data to make better and more informed decisions. The disclosed digital platform can also offer platform-side computational benefits by reducing costs via the elimination of waste and inefficiency that is so prevalent throughout healthcare experiences.

The disclosed digital platform can be health plan-driven and consumer-focused and can include various components that manage a user's health, network, and money. The disclosed platform contains the functions necessary for consumers to manage various aspects of their health and healthcare experiences. The disclosed platform can be triangular in nature: for example, the platform can connect the consumers, providers, and the corresponding health plans on a single platform. This provides transparency, knowledge, and control to consumers while managing their own healthcare. For example, consumers can find providers and service locations, perform appointment scheduling, and acquire telemedicine options, as well as manage prescriptions, medical records, and medical alerts/reminders.

In addition, the disclosed platform can operate in real-time; for example, the platform can utilize personalized Quick Response (QR) codes (also referred to herein as PQR codes), which are three-dimensional barcodes that can store more data and various additional types of data (including images) than two-dimensional barcodes. PQR codes as used herein can improve the storage and retrieval of health plan coverage and personal medical information and can facilitate the exchange of information in real-time. By scanning a PQR code (e.g., that operates as a health benefits “smart card”) at the time of service, both consumers and providers obtain medical benefits, eligibility information, financial responsibility confirmation, medical eligibility alerts, a priori authorization requirements, etc.

Furthermore, the disclosed digital platform can add significant benefits to consumers at the “first mile” (shopping and selecting) and the “last mile” (payment) for healthcare experiences; these instances are often pain points for healthcare consumers.

Other benefits of the disclosed digital platform can include the improvement of the selection and delivery of care, clinical outcomes, cost-effectiveness of care, and the consumer's overall healthcare experience. In addition, the disclosed digital platform can: 1) provide an auto-populated portable medical record for services rendered and with corresponding results regardless of provider-affiliation; 2) provide a better understanding of consumers' financial obligations prior to scheduling treatments; 3) compare treatment costs; 4) schedule treatment appointments; and 5) verify treatment eligibility and coverage prior to and/or at point-of-service.

Further technical advantages of the disclosed platform include confirmation that pre-authorizations have been obtained prior to treatment; tracking of prescription refills; tracking of deductible and out-of-pocket expenses; enabling of medical claims payments; elimination of unnecessary and duplicate medical services; providing of real-time communication capabilities (voice, text, email, etc.); disease management protocols; real-time feedback and satisfaction mechanisms; creates “peer2peer” consumer platform that share personal experiences; and directs consumers to the “best place” for care by utilizing comprehensive data analytics and artificial intelligence to perform predictive analysis.

With respect to waste reduction, the Journal of the American Medical Association (JAMA) estimates there is up to $192 billion wasted annually on transaction processing and administrative complexities in the healthcare industry. There is a tremendous amount of inaccurate and incomplete information associated with the transaction process, which greatly contributes to this waste. By consumers scanning their smart card at the time of service, providers will be able to verify current eligibility, determine whether scheduled procedures require pre-authorization (and if so, have they been obtained), and obtain accurate patient co-insurance/deductible, etc.) on a real-time basis. Another benefit of consumers scanning their smart card at the time of service is that providers will be able to verify that the consumers are medically eligible for the scheduled procedures, as well as to confirm if consumers have had the same tests recently performed. For example, if a consumer has a pacemaker, they are not eligible to undergo an MRI test. In another example, if a consumer is allergic to iodine, they are not eligible to undergo a CT scan with contrast.

In addition, the disclosed platform can use medical claims data to automatically populate a consumer's medical record and digitize the results for a variety of diagnostic tests (e.g., labs, imaging, EKG, pathology, etc.), pair the results with the specific procedure, and automatically populate the corresponding medical record. In other words, consumers can utilize a true portable medical record across all providers regardless of healthcare-system affiliation. This also allows for more transparent management of expenses (e.g., deductibles, out-of-pocket expenses, year-to-date costs, service cost calculators, claim statuses, etc.).

Benefits to health plan managing systems can include reduced administrative costs as a result of more timely and accurate claims processing, improved clinical results, the elimination of redundant services with real-time interfaces, and the building of customer loyalty and retention.

Benefits to healthcare providers can include the better management of services and treatments that require pre-authorizations and of patient financial responsibilities at the time of service, the reduction of unnecessary and duplicate tests, and the reduction of medical errors.

In some embodiments, the disclosed digital platform can flag a procedure at the time of service if the required authorization has not been obtained, provide consumers and their providers with relevant information at the time of service such as any monetary out-of-pocket obligations, alert providers if the consumer is not medically eligible, and provide consumers with multiple treatment locations for a specific procedure along with appointment options and payment requirements.

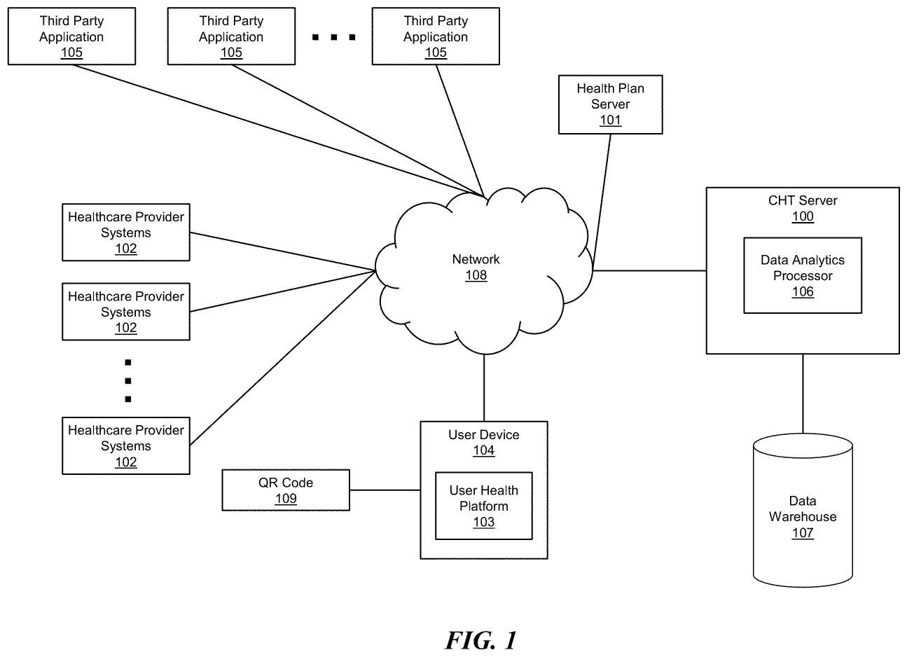

FIG. 1 is a block diagram of an example system for providing a comprehensive digital healthcare platform according to some embodiments of the present disclosure. In one or more embodiments, the system can include CHT server 100 and data warehouse 107. CHT server 100 includes a data analytics processor 106 and interfaces with a user's health benefits and plan that is maintained e.g., on a health plan server 101. In some embodiments, health plan server 101 stores and maintains health benefits and plans for a plurality of users. CHT server 100 also interfaces with a plurality of healthcare provider systems 102 (generally referred to herein as a “healthcare provider system 102” or collectively referred to herein as “healthcare provider systems 102”). CHT server 100 can interact with health plan server 101 and one or more of the healthcare provider systems 102 in a novel arrangement to create a user health platform 103 that is maintained on a user device 104. CHT server 100 can interact with the various components of the system via a network 108. In addition, CHT server 100 and the user device 104 can interact with one or more third party applications 105. In one or more embodiments, CHT server 100 provides real-time processing by using a personalized health management quick response (QR) code 109 that may be used as a health benefits “smart card.” In some embodiments, the QR code 109 can be a 3D QR code. The QR code 109 and processing disclosed herein transforms the storage and retrieval of a user's health coverage and benefits and personal medical information and facilitates the exchange of information on a real-time basis. The system of FIG. 1 utilizes a high-level of sophisticated knowledge to coordinate the integration of various separate and independent computer systems in conjunction with the utilization of 3D QR technology.

The network 108 can include one or more wide areas networks (WANs), metropolitan area networks (MANs), local area networks (LANs), personal area networks (PANs), or any combination of these networks. The network 108 can include a combination of one or more types of networks, such as Internet, intranet, Ethernet, twisted-pair, coaxial cable, fiber optic, cellular, satellite, IEEE 801.11, terrestrial, and/or other types of wired or wireless networks. The network 108 can also use standard communication technologies and/or protocols.

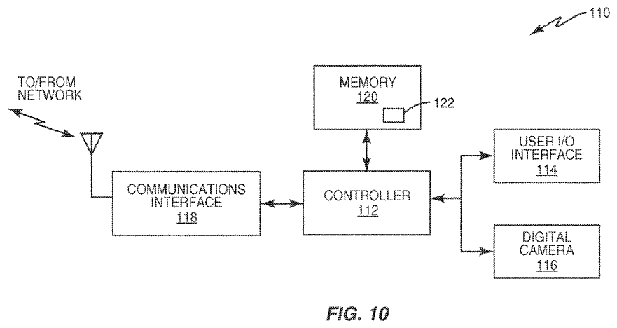

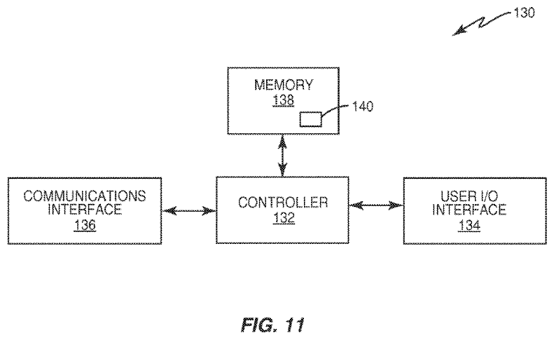

A user device 104 can include one or more computing devices capable of receiving user input, transmitting and/or receiving data via the network 108, and or communicating with CHT server 100. In some embodiments, a user device 104 can be a conventional computer system, such as a desktop or laptop computer. Alternatively, a user device 104 can be a device having computer functionality, such as a personal digital assistant (PDA), a mobile telephone, a smartphone, tablet or other suitable device. In some embodiments, a user device 104 can be the same as or similar to the computing device described below with respect to FIGS. 10 and 11. In some embodiments, the system can include any number of user devices 104.

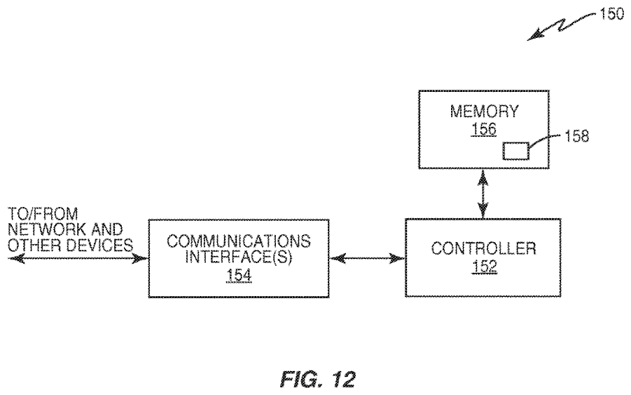

CHT server 100 may include any combination of one or more of web servers, mainframe computers, general-purpose computers, personal computers, or other types of computing devices. CHT server 100 may represent distributed servers that are remotely located and communicate over a communications network, or over a dedicated network such as a local area network (LAN). CHT server 100 may also include one or more back-end servers for carrying out one or more aspects of the present disclosure. In some embodiments, CHT server 100 may be the same as or similar to the computing device described below in the context of FIG. 12.

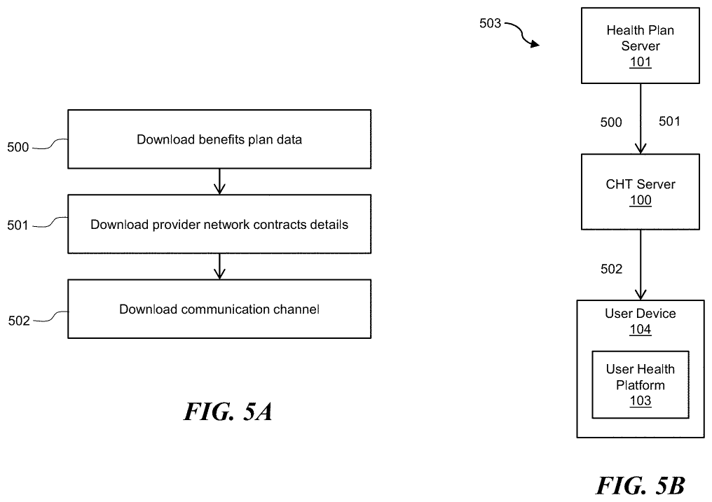

In one or more embodiments disclosed herein, the platform (e.g., the system of FIG. 1) uses a novel application of a 3D QR code 109 to establish various communication channels (FIGS. 5-9) between a user device 104 and various separate and independent computer systems, such as computer systems owned/operated by health plan server 101, healthcare provider systems 102, etc., that contain relevant and interrelated data that either belongs to the user or that the user needs in order to complete a necessary health care function. This can prove especially useful for hand-held devices in that the user can perform various interrelated functions without the need to constantly log in and out of multiple computer systems to successfully perform various functions.

In one or more embodiments, CHT server 100 can be configured to obtain various information from the health plan server 101 related to a user (e.g., the user of user device 104), such as a health plan or health benefits contract. This information can include medical and prescription benefits, one or more providers' negotiated fee schedules, prior authorization requirements, and co-insurance and deductible information. CHT server 100 can use this information to populate and maintain a medical record for the user.

CHT server 100 can also obtain various information from healthcare provider systems 102, such as profiles of providers that are both in and out of network, service locations, and telemedicine options. In addition, CHT server 100 can interact with the healthcare provider systems 102 to facilitate claims processing, obtain medical results (e.g., labs, tests, imaging, etc.). In some embodiments, medical results can also be obtained from third party applications 105. In one or more embodiments, CHT server 100 can maintain various health management applications, disease management applications, and a medical library for access and use by user device 104.

In some embodiments, user device 104 can interact with one or more healthcare provider systems 102 to access a communication hub and manage email addresses, text messaging, telemedicine functionality, and an appointment scheduler.

CHT server 100 compiles the information received and maintains it in a user health platform 103 provided on the user device 104. In one or more embodiments, the user health platform 103 maintains health information for the user, network information for the user, and money information for the user. The health information can include medical records, disease management protocols, medical alerts/reminders, and prescription information. The network information can include providers (both in and out of network), service locations, appointment scheduling information, telemedicine options, and a communications hub. The money information can include plan benefits, premium payment information, claims statuses, a service cost calculator, pre-authorization prompts, and year-to-date spend balances.

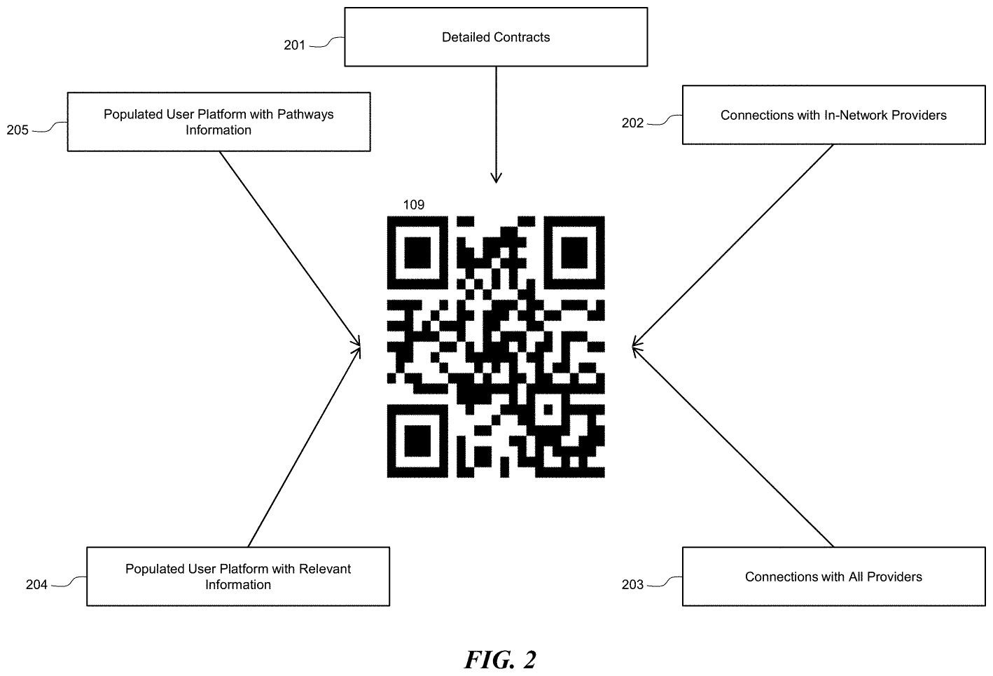

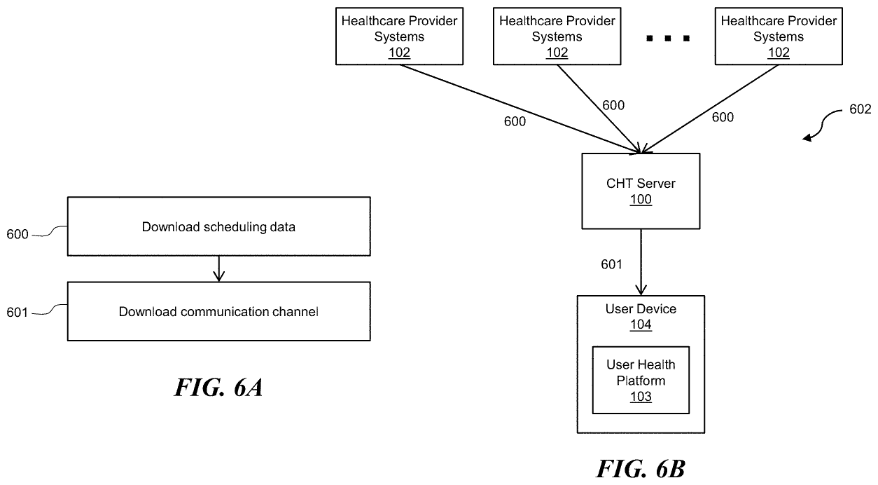

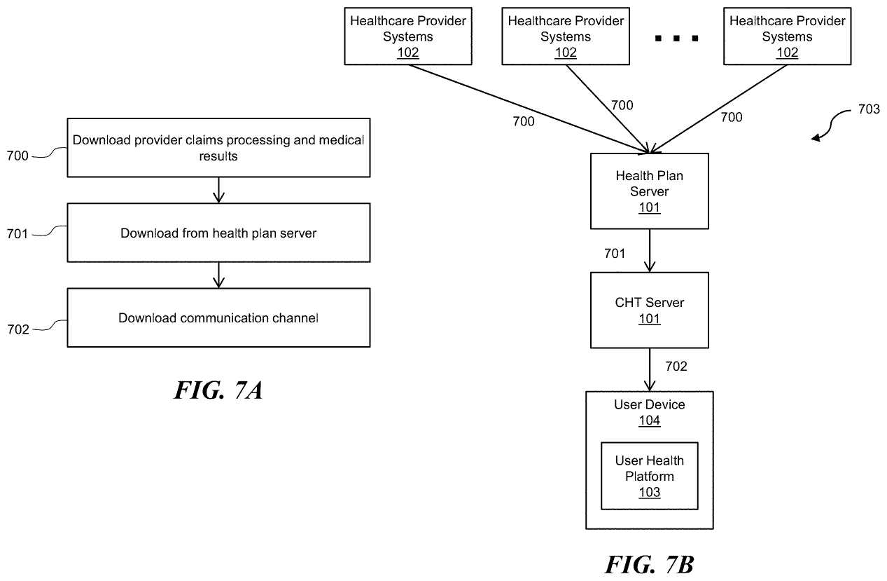

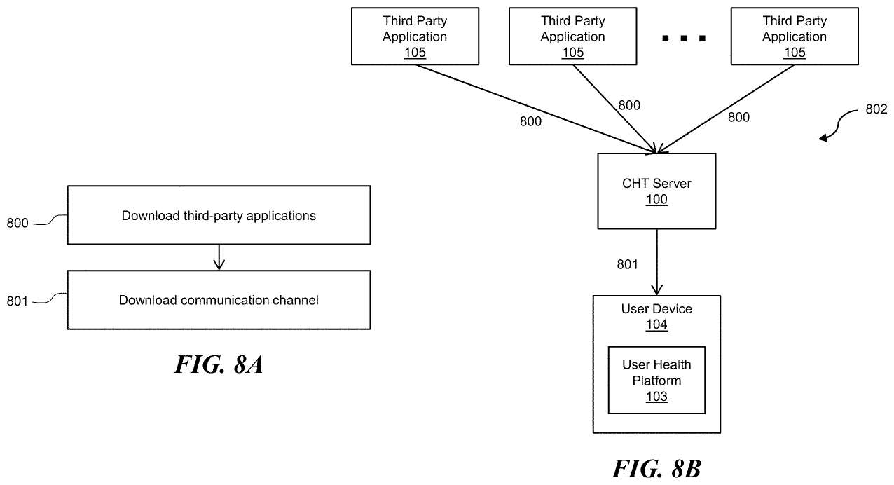

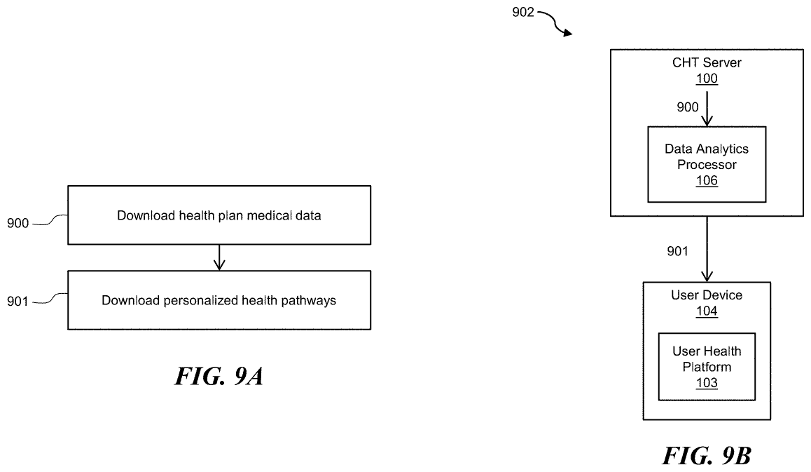

FIG. 2 shows an example transformative 3D health management QR code 109 according to some embodiments of the present disclosure. As noted above, the QR code 109 can operate as a “smart card” for a user and includes various information embedded into the code that is obtained from communication channels, such as detailed contract information 201 (see FIGS. 5A and 5B), information on connections with in-network providers 202 (see FIGS. 6A and 6B), information on connections with all providers 203 (see FIGS. 7A and 7B), a populated user platform 204 with relevant information (see FIGS. 8A and 8B), and a populated user platform 205 with health pathways information (see FIGS. 9A and 9B).

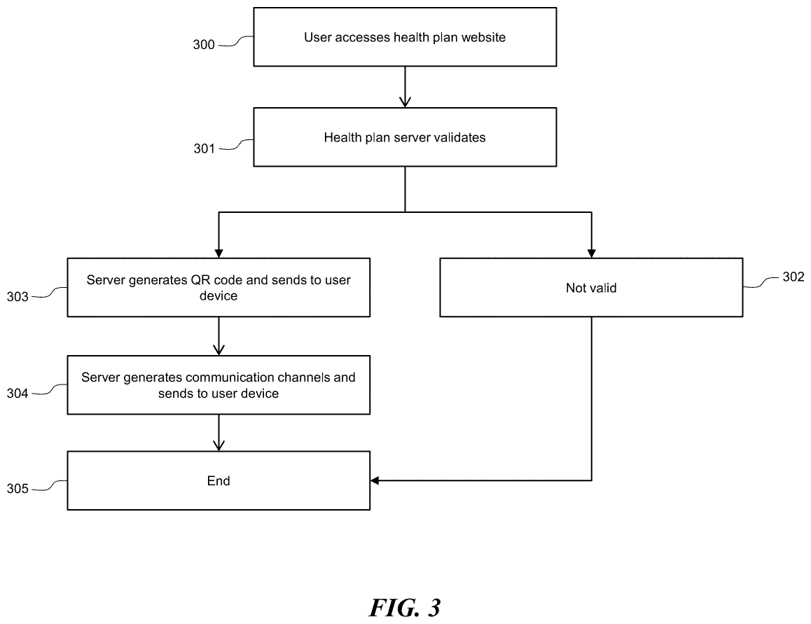

FIG. 3 is a flow diagram showing example processing that can launch a digital healthcare platform according to some embodiments of the present disclosure. In some embodiments, the process of FIG. 3 is performed to authenticate a user device (e.g., user device 104) so that the user is able to perform protected functions, such as logging into desired websites and authorizing payments for various transactions. At block 300, a user accesses a health plan website (or other similar entity) and logs into the website. In one or more embodiments, the user can access the website and log in via user device 104. At block 301, the health plan website (e.g., operating on health plan server 101) validates the user's identity. If the user's identity cannot be validated at 302, the process terminates at block 305. If the user's identity is validated (block 303), CHT server 100 generates the QR code 109 and transmits it to the user device 104. At block 304, CHT server 100 generates various communication channels and sends them to the user device 104. Additional details on the communication channels are described with respect to FIGS. 5A-9B.

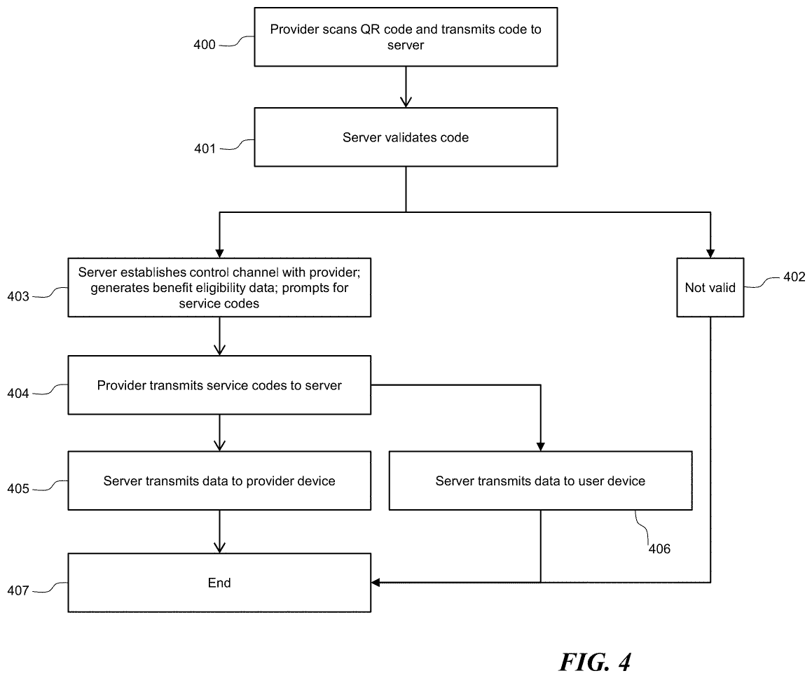

FIG. 4 is a flow diagram showing example processing that can be performed at a point-of-service according to some embodiments of the present disclosure. In one or more embodiments, the process of FIG. 4 is performed when a QR code 109 is displayed on user device 104 and allows the user, provider, and or health plan manager to retrieve, communicate, and process real-time information as disclosed herein. At block 400, a provider (e.g., via a provider device part of health care provider system 102) scans the QR code 109 and transmits the QR code 109 to CHT server 100. At block 401, CHT server 100 validates the QR code 109. If the code is determined to be invalid at block 402, the process terminates at block 407.

If the QR code 109 is validated by CHT server 100, then processing advances to block 403. At block 403, CHT server 100 establishes a control channel with one or more healthcare provider systems 102, generates benefit eligibility data, and provides a prompt for one or more service codes. At block 404, the provider enters the relevant code or codes and transmits it/them to CHT server 100. In some embodiments, the one or more codes can include various service codes such as preliminary diagnostic and procedure codes. At block 405, CHT server 100 transmits various data to the provider device. The data can include medical eligibility information, medical complications (if any), pre-authorization requirements (if any), medical redundancy information, medical necessity information, patient financial responsibility information (e.g., co-insurance and deductible information), etc. At block 406, CHT server 100 transmits the same data to user device 104. A benefit of the process of FIG. 4 is that, by scanning the QR code 109 at the time of service, both the user and provider obtain certain relevant information that is normally not easily accessible.

FIGS. 5A and 5B show an example communication channel 503 (including its processing in FIG. 5A and the communications between devices in FIG. 5B) according to some embodiments of the present disclosure. The communication channel can facilitate the transfer of detailed contract information (e.g., information 201 of FIG. 2). At block 500, CHT server 100 downloads benefits plan data from the health plan server 101. Benefits plan data can include prior authorization requirements, co-insurance requirements, and deductible requirements. At block 501, CHT server 100 downloads provider network contracts details from health plan server 101. Provider network contracts details can include contracted rates (e.g., by procedure and acceptable diagnosis codes or by provider and service location) and telemedicine options. At block 502, user device 104 can download the benefits plan data and the provider network contracts details onto the user health platform 103.

FIGS. 6A and 6B show another example communication channel 602 (including its processing in FIG. 6A and the communications between devices in FIG. 6B) according to some embodiments of the present disclosure. The communication channel can facilitate the transfer of information on the connections with in-network providers (e.g., information 202 of FIG. 2). At block 600, CHT server 100 downloads scheduling data from one or more healthcare provider systems 102. Scheduling data can include schedules and communication hubs from in-network providers. At block 601, user device 104 downloads the communication channel so that the user health platform 103 has access to in-network providers, service locations, appointment scheduling, and telemedicine options.

FIGS. 7A and 7B show another example communication channel 703 (including its processing in FIG. 7A and the communications between devices in FIG. 7B) according to some embodiments of the present disclosure. The communication channel can facilitate the transfer of information on connections with all providers (e.g., information 203 of FIG. 2). At block 700, health plan server 101 downloads provider claims processing information and medical results from one or more health care provider systems 102. At block 701, CHT server 101 downloads the claims processing information and medical results from the health plan server 101. At block 702, user device 104 downloads the communication channel so that the user health platform 103 has access to health information (medical records, disease management protocols, medical alerts and reminders, prescriptions, and a communication hub) and money information (plan benefits, premium payment, claims statuses, a service cost calculator, pre-authorization prompts, and year-to-date spend balances).

FIGS. 8A and 8B show another example communication channel 802 (including its processing in FIG. 8A and the communications between devices in FIG. 8B) according to some embodiments of the present disclosure. The communication channel can facilitate the transfer of information on a populated platform with relevant information (e.g., platform 204 of FIG. 2). At block 800, CHT server 100 downloads one or more third party applications 105, such as health management applications, disease management applications, and medical library applications. At block 801, user device 104 downloads the communication channel so that the user health platform 103 has access to health information (medical records, disease management protocols, medical alerts and reminders, prescriptions, and a communication hub).

FIGS. 9A and 9B show another example communication channel 902 (including its processing in FIG. 9A and the communications between devices in FIG. 9B) according to some embodiments of the present disclosure. The communication channel can facilitate the transfer of information on a populated platform with health pathways information (e.g., platform 204 of FIG. 2). At block 900, the data analytics processor 106 downloads health plan medical data from CHT server 100. The health plan medical data can include medical results. At block 901, user device 104 downloads personalized health pathways from CHT server 100.

FIG. 10 is a block diagram illustrating some of the components of a computing device 110 suitable for a user device and or provider device configured according to one embodiment of the present disclosure. Device 110 may be a cellular telephone or smartphone. However, this is for illustrative purposes only. The present disclosure is also suitable for use in other communications devices including, but not limited to, satellite telephones, Personal Digital Assistants (PDAs), and computing devices such as laptop and notebook computers, and tablet computing devices.

As seen in FIG. 10, device 110 comprises a controller 112, user I/O interface 114, digital camera circuitry 116, one or more communications interfaces 118, and a memory 120. Controller 112 may be, for example, one or more general purpose or special purpose microprocessors that control the operation and functions of device 110 in accordance with program instructions and data stored in memory 120. In one embodiment of the present disclosure, the controller 112 executes an image processing program 122 to analyze and extract parameters from and image of a PQR code. Based on this information, controller 112 is programmed to establish a communications channel with server 150 and/or device 130.

User I/O interface 114 enables a user to input information into device 110 and includes devices and controls that facilitate such interaction. Typically, the user I/O interface 114 includes a display (e.g., a touch-sensitive display) that allows the user to view information such as dialed digits, images, call status, menu options, and other service information. In some embodiments, the user I/O interface 114 may also include a keypad that allows the user to enter digits and other alpha-numeric input. The camera circuitry 116 functions to capture images of objects such as PQR codes.

Although not specifically seen in FIG. 10, the digital camera circuitry 116 generally includes components such as a lens assembly, an image sensor, and an image processor. In operation, light entering the lens assembly is focused onto the image sensor, which may be, for example, a charge-coupled device (CCD) or a complementary metal oxide semiconductor (CMOS) image sensor. The image processor, if present, then processes the raw image data captured by the image sensor for subsequent storage in memory 120 or for output to a display. According to the present disclosure, the image processor analyzes the captured image of the PQR code and extracts the parameters embedded therein. The parameters are then sent to the communications interface for transmission to a server 150.

The communications interface 118 may be a fully functional cellular radio transceiver for transmitting signals to and receiving signals from a base station or other access node in a wireless communications network. Those skilled in the art will appreciate that the communications interface 118 may implement any one of a variety of communication standards including, but not limited to, the standards known as the Global System for Mobile Communications (GSM), the General Packet Radio Service (GPRS), cdma2000, Universal Mobile Telecommunications System (UMTS), Wideband Code Division Multiple Access (WCDMA), 3GPP Long Term Evolution (LTE), Worldwide Interoperability for Microwave Access (WiMAX), and Wireless Local Area Network (WLAN) or other 802.11x interface.

In addition to, or in lieu of, the communications interface being a cellular transceiver, the communications interface may also include a short-range transceiver operating according to the BLUETOOTH protocol. As is known in the art, BLUETOOTH is a universal radio interface that enables two or more wireless devices to communicate wirelessly via short-range ad hoc networks. As discussed previously, the device 110 may establish a direct, short-range communication link with device 130 using this short-range communication transceiver.

FIG. 11 illustrates some of the components of a computing device 130 that may be used in the system illustrated in FIG. 1. Device 130 may be a personal computing device, or a web-enabled TV. However, the present disclosure is not limited to these embodiments. In other embodiments for example, the device 130 is a tablet computing device.

Device 130 generally comprises a controller 132, a memory 138, a user I/O interface 134, and a communications interface 136. The operation of these components is similar to those described previously. Therefore, they will not be discussed further here. It is sufficient, however, to note that the memory 138 comprises a browser application 140 that, when executed by controller 132, causes device 130 to generate and transmit certain messages to a server within the system (details of a server 150 are discussed with reference to FIG. 12). Specifically, the browser application 140 may generate HTTP GET messages to request a random number from server 150 whenever the user enters the URI of a web site. The HTTP GET message is the trigger that causes the server to generate the parameters for the PQR code and respond with an HTTP message containing the QR code for display. When device 130 is a provider device, for example, it may also contain a bar code reader or camera circuitry 116 functions to capture images of objects such as PQR codes.

FIG. 12 is a block diagram illustrating some of the components of the server 150 configured according to one embodiment of the present disclosure. As seen in FIG. 12, server 150 comprises, among other things, a controller 152, communications interface 154, and memory 156. The memory 156 may further store a software module 158. The controller 152 and communications interface 154 operate as previously described. However, the software module 158 configures controller 152 of server 150 to generate the parameters and the PQR code, as previously described. In addition to generating these artifacts in response to a request from device 130, software module 158 also causes the controller 152 to generate new parameters and PQR codes whenever the user performs a predefined action from device 110 or device 130.

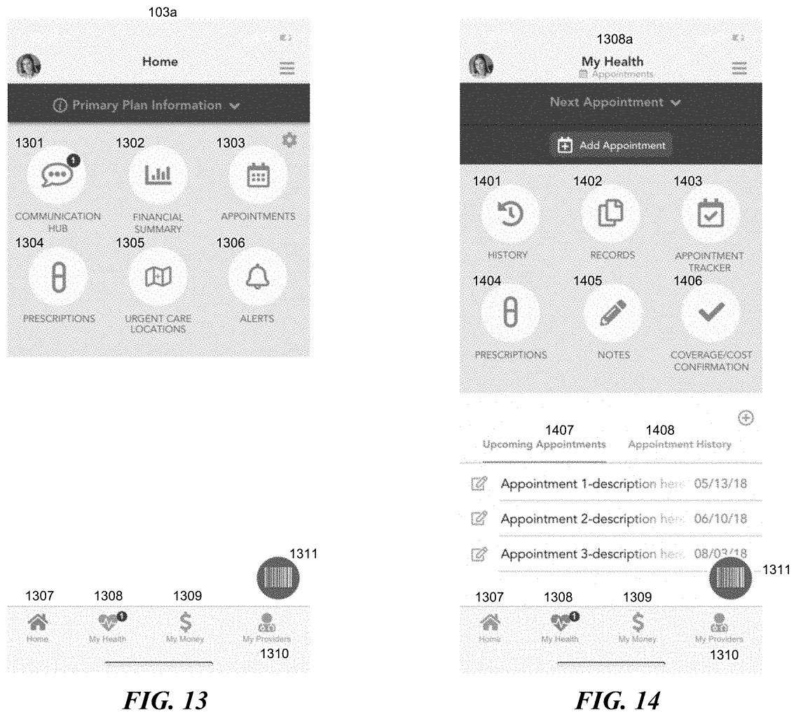

FIG. 13 is an example user interface 103a that can be presented on a user device according to some embodiments of the present disclosure. In one or more embodiments, the interface 103a can be part of the user health platform 103 that is viewable by a user on the user device 104. The interface 103a can include various buttons to access certain features, such as a communication hub 1301, a financial summary 1302, appointments 1303, prescriptions 1304, urgent care locations 1305, and alerts 1306 (e.g., appointment reminder, payment reminders, prescription reminders, etc.). In addition, the user can access a home tab 1307, a My Health tab 1308, a My Money tab 1309, a My Providers tab 1310, and a button 1311 to display the QR code 109.

FIG. 14 is another example user interface 1308a that can be presented on a user device according to some embodiments of the present disclosure. The interface 1308a can be displayed when a user selects the My Health tab 1308. The My Health interface 1308a can include various buttons to access certain features, such as a history of transactions 1401, medical records 1402, an appointment tracker 1403, prescriptions 1404, notes 1405, and coverage/cost information 1406. In addition, My Health interface 1308a can include a list 1407 of upcoming appointments and an appointment history 1408.

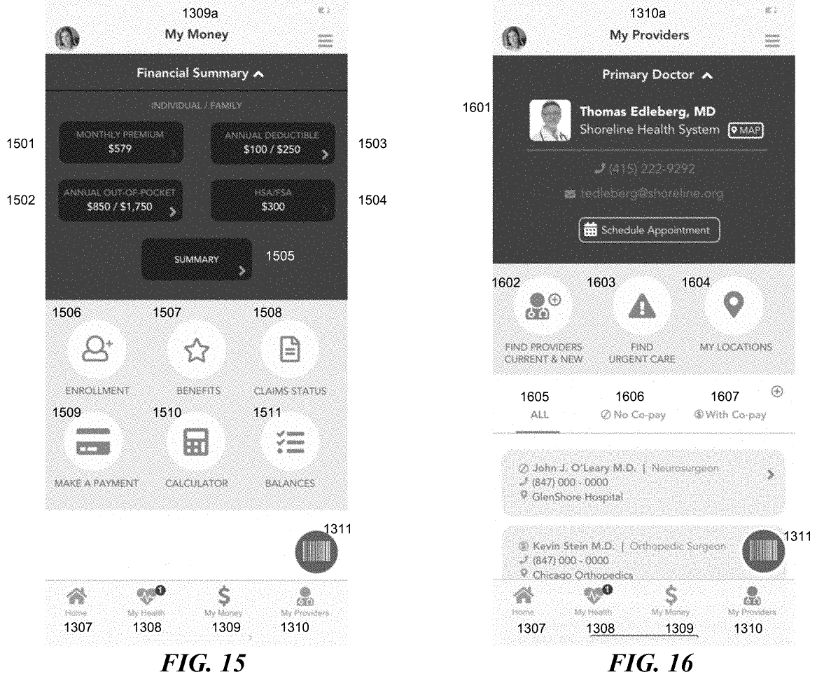

FIG. 15 is another example user interface 1309a that can be presented on a user device according to some embodiments of the present disclosure. The interface 1309a can be displayed when a user selects the My Money tab 1309. My Money interface 1309a can include a financial summary that includes a monthly premium 1501, annual out-of-pocket maximums 1502, annual deductibles 1503, HSA/FSA information 1504, and a summary button 1505. In addition, My Money interface 1309a can include various buttons to access certain features, such as enrollment information 1506, benefits information 1507, claim status information 1508, payment making features 1509, a calculator 1510, and balances information 1511.

FIG. 16 is another example user interface 1310a that can be presented on a user device according to some embodiments of the present disclosure. The interface 1310a can be displayed when a user selects the My Providers tab 1310. My Providers interface 1310a can include a summary 1601 of the user's primary doctor. My Providers interface 1310a can also include various buttons to access certain features, such as a button 1602 to find current and new providers, a button 1603 to find urgent care, and a button 1604 to display the users saved locations. In addition, My Providers interface 1310a also includes a list 1605 of all providers, a list 1606 of providers with no co-pay, and a list 1607 of providers with co-pay.

It should be appreciated, however, that the examples set forth herein are provided merely for the purpose of explanation and are in no way to be construed as limiting. While reference to various embodiments is made, the words used herein are words of description and illustration, rather than words of limitation. Further, although reference to particular means, materials, and embodiments are shown, there is no limitation to the particulars disclosed herein. Rather, the embodiments extend to all functionally equivalent structures, methods, and uses, such as are within the scope of the appended claims.

1. A digital healthcare system comprising:
a server communicably coupled to a user device associated with a user, the server being configured to:
obtain information associated with the user by:
downloading benefits plan data and provider network contract details from a health plan server;
downloading scheduling data from one or more provider devices;
downloading medical results associated with the user and claims processing information from the health plan server; and
downloading one or more third-party applications;
embed the information into a QR code transmitted to the user device; and
establish one or more communication channels for the user device.

2. The digital health system of claim 1, wherein the server is further configured to:
receive data corresponding to a scanning of the QR code from at least one of the one or more provider devices;
validate the QR code based on the received data; and
generate eligibility data for the user.

3. The digital health system of claim 2, wherein the server is further configured to:
transmit a service code prompt to the at least one of the one or more provider devices;
receive a service code from the at least one of the one or more provider devices; and
transmit, to the at least one of the one or more provider devices, at least one of medical eligibility information associated with the user, medical complications information associated with the user, medical necessity information associated with the user, co-insurance information associated with the user, or deductible information associated with the user.

4. The digital health system of claim 3, wherein the server is further configured to:
in response to receiving the service code, transmit to the user device at least one of the medical eligibility information associated with the user, the medical complications information associated with the user, the medical necessity information associated with the user, the co-insurance information associated with the user, or the deductible information associated with the user.

5. The digital health system of claim 1, wherein the one or more third-party applications comprise at least one of a health management application, a disease management application, or a medical library application.

6. The digital health system of claim 5, wherein one of the communication channels allows the user device to download at least one of medical records information, disease management protocol information, medical reminder information, or prescription information.

7. The digital health system of claim 6, wherein the at least one of medical records information, disease management protocol information, medical reminder information, or prescription information is maintained in and accessible via an application on the user device.

8. The digital health system of claim 1, wherein the QR code comprises a medical record for the user populated with medical testing information.

9. The digital health system of claim 8, wherein the server is configured to identify medically inappropriate services for the user based on the medical record.

10. The digital health system of claim 1, wherein the server is configured to flag a procedure at a time of service if a pre-authorization has not been obtained for the user.

11. A method, performed by a server communicably coupled to a user device associated with a user, comprising:
obtaining information associated with the user by:
downloading benefits plan data and provider network contract details from a health plan server;
downloading scheduling data from one or more provider devices;
downloading medical results associated with the user and claims processing information from the health plan server; and
downloading one or more third-party applications;
embedding the information into a QR code transmitted to the user device; and
establishing one or more communication channels for the user device.

12. The method of claim 11 further comprising:
receiving data corresponding to a scanning of the QR code from at least one of the one or more provider devices;
validating the QR code based on the received data; and
generating eligibility data for the user.

13. The method of claim 12 further comprising:
transmitting a service code prompt to the at least one of the one or more provider devices;
receiving a service code from the at least one of the one or more provider devices; and
transmitting, to the at least one of the one or more provider devices, at least one of medical eligibility information associated with the user, medical complications information associated with the user, medical necessity information associated with the user, co-insurance information associated with the user, or deductible information associated with the user.

14. The method of claim 13 further comprising:
in response to receiving the service code, transmitting to the user device at least one of the medical eligibility information associated with the user, the medical complications information associated with the user, the medical necessity information associated with the user, the co-insurance information associated with the user, or the deductible information associated with the user.

15. The method of claim 14, wherein the one or more third-party applications comprise at least one of a health management application, a disease management application, or a medical library application.

16. The method of claim 15, wherein one of the communication channels allows the user device to download at least one of medical records information, disease management protocol information, medical reminder information, or prescription information.

17. The method of claim 16, wherein the at least one of medical records information, disease management protocol information, medical reminder information, or prescription information is maintained in and accessible via an application on the user device.

18. The method of claim 11, wherein the QR code comprises a medical record for the user populated with medical testing information.

19. The method of claim 18 further comprising identifying medically inappropriate services for the user based on the medical record.

20. The method of claim 11 further comprising flagging a procedure at a time of service if a pre-authorization has not been obtained for the user.
